(19) United States
(12) Patent Application Publication
(10) Pub. No.: US 2026/0061200 A1
(43) Pub. Date: Mar. 5, 2026

(54) MULTI-DIRECTIONAL TRIGGER REACTIVE SCHEDULING

(71) Applicant: Boston Scientific Neuromodulation Corporation, Valencia, CA (US)

(72) Inventor: Lisa Denise Moore, Glendale, CA (US)

(73) Assignee: Boston Scientific Neuromodulation Corporation, Valencia, CA (US)

(21) Appl. No.: 19/312,173

(22) Filed: Aug. 27, 2025

Related U.S. Application Data
(60) Provisional application No. 63/688,511, filed on Aug. 29, 2024.
(60) Provisional application No. 63/776,783, filed on Mar. 24, 2025.

(51) Int. Cl. A61N 1/36

(52) U.S. Cl. CPC A61N 1/36139; A61N 1/36132

(57) ABSTRACT

Methods and systems for delivering a neuromodulation therapy. For a neuromodulation system implanted in a patient, trigger parameters including multiple trigger conditions and responses may be set. The trigger parameters may include a first trigger window condition with a first trigger response and a second trigger window condition with a second trigger response. Upon reaching a trigger window start time, observation for the trigger conditions may be initiated. In response to observing the first trigger window condition, the first trigger response may be executed to change the neuromodulation therapy delivered to the patient. In response to observing the second trigger window condition, the second trigger response may be executed. The trigger conditions may include user inputs, and/or sensed signals from internal or external sensors, while responses may include changes to stimulation parameters. Patient verification may be required before implementing therapy changes based on observed trigger conditions.

### CROSS-REFERENCE TO RELATED APPLICATIONS

This application claims the benefit of priority under 35 U.S.C. § 119 to U.S. Provisional Application Ser. No. 63/688,511, filed Aug. 29, 2024 and to U.S. Provisional Application Ser. No. 63/776,783, filed Mar. 24, 2025, the disclosures of which are incorporated herein by reference.

### TECHNICAL FIELD

The disclosure is directed to programming and controlling deep brain stimulation systems and/or spinal cord stimulation systems. More particularly, the disclosure is directed to methods and systems for a creation and activation of stimulation schedules.

### BACKGROUND

Implantable and/or wearable stimulation systems for the treatment of various diseases and disorders of the neurological system have proven effective in a wide variety of ways. For example, spinal cord stimulation (SCS) systems are accepted treatments for chronic pain syndromes while deep brain stimulation (DBS) systems may be used for the treatment of dementia, Alzheimer's disease, Parkinson's disease, dyskinesias, tremors, depression, anxiety or other mood disorders, sleep related conditions, etc. An SCS or DBS system typically includes an Implantable Pulse Generator (IPG). The IPG is coupled to tissue-stimulating electrodes carried on the distal end of one or more electrode leads that are implanted near the spinal column. The proximal ends of the one or more leads are tunneled through the patient's tissue to a location where the IPG is implanted. The proximal ends of the leads are coupled to the IPG to provide electrical stimulation from the electrodes to provide therapeutic benefits.

In some cases, the IPG may be configured to deliver therapy in accordance with one or more predefined stimulation programs. The predefined stimulation programs may be scheduled to delivery therapy at varying levels (intensities) at varying times of the day. The stimulation programs may operate on a schedule that is irrespective of the patient's local time or may occur in a time bound manner to sync with expected events such as, but not limited to, sleep or scheduled medications. However, patients may travel outside of their home time zones, sleep at different hours, or take their medications at varying times of the day. Thus, an ideal time or event-based stimulation schedule may be difficult to obtain as an estimate of fixed time points (e.g., sleep, medication, or the like) are inaccurate even for a same patient due to common life circumstances. New and alternative methods and devices which allow for prescheduled program changes of the IPG may be desired.

### Overview

A first illustrative and non-limiting example takes the form of a method of delivering a neuromodulation therapy comprising: setting, for a neuromodulation system implanted in a patient, a trigger window and a trigger requirement: the trigger window including a trigger window start time, a trigger window end time, and a definition of a trigger window trigger, and the trigger requirement including a trigger requirement start time, a trigger requirement end time, and a definition of a trigger requirement trigger; upon reaching the trigger window start time: initiating observation for the trigger window trigger; in response to observation of the trigger window trigger, changing a neuromodulation therapy delivered to the patient; and upon reaching the trigger requirement start time: initiating observation for the trigger requirement trigger; and in response to observation of the trigger requirement trigger, changing the neuromodulation therapy delivered to the patient.

Additionally or alternatively, the definition of a trigger window trigger includes a definition of a first trigger window condition and a first trigger response, and a definition of a second trigger window condition and a second trigger response; the step of initiating observation for the trigger window trigger includes observing for each of the first trigger window condition and the second trigger window condition; the step of changing a neuromodulation therapy delivered to the patient in response to observation of the trigger window trigger comprises either: in response to observing the first trigger window condition, executing the first trigger response to change the neuromodulation therapy; or in response to observing the second trigger window condition, executing the second window trigger response to change the neuromodulation therapy; the definition of a trigger requirement trigger includes a definition of a first trigger requirement condition and a first trigger requirement response, and a definition of a second trigger requirement condition and a second trigger requirement response; initiating observation for the trigger requirement trigger includes observing for each of the first trigger requirement condition and the second trigger requirement condition; and the step of changing the neuromodulation delivered to the patient therapy in response to observation of the trigger requirement trigger comprises either: in response to observing the first trigger requirement condition, executing the first trigger requirement response to change the neuromodulation therapy; or in response to observing the second trigger requirement condition, executing the second trigger requirement response to change the neuromodulation therapy.

Additionally or alternatively, each of the first trigger window condition and the second trigger window condition is at least one of: a user input received through a patient device; a sensed signal from an internal sensor of the neuromodulation system; or a sensed signal from an external sensor; and the first trigger response and the second trigger response are each: an amplitude change; a frequency change; a pulse width change; a fractionalization change; a program change; or a combination thereof.

Additionally or alternatively, each of the of the first trigger requirement condition and the second trigger requirement condition is at least one of: a user input received through a patient device; a sensed signal from an internal sensor of the neuromodulation system; or a sensed signal from an external sensor; and the first trigger requirement response and the second trigger requirement response are each: an amplitude change; a frequency change; a pulse width change; a fractionalization change; a program change; or a combination thereof.

Additionally or alternatively, changing the neuromodulation therapy further comprises: prior to implementing the first trigger response or the second window response, sending a notification to a patient device that the first trigger window condition or the second trigger window condition has been detected; and implementing the neuromodulation therapy change upon receiving a patient approval on the patient device.

Additionally or alternatively, changing the neuromodulation therapy further comprises: prior to implementing the first trigger requirement response or the second trigger requirement response, sending a notification to a patient device that the first trigger requirement condition or the second trigger requirement condition has been detected; and implementing the therapy change upon receiving a patient approval on the patient device.

Additionally or alternatively, changing the neuromodulation therapy comprises in response to not receiving a patient approval through the patient device, maintaining the current stimulation parameters.

Additionally or alternatively, the step of changing the neuromodulation therapy further comprises: detecting the first trigger window condition and the second trigger window condition during the same trigger window; for each detected trigger window condition: identifying the trigger response for each trigger window condition; sending a trigger-specific alert for each trigger window condition to the patient device providing information about each identified trigger window condition and the trigger response to be implemented; receiving an individual patient approval or rejection through the patient device for each trigger response; and implementing the trigger responses that received the individual patient approval.

Additionally or alternatively, the step of changing the neuromodulation therapy further comprises: detecting the first trigger requirement condition and the second trigger requirement condition during the same trigger window; for each detected trigger requirement condition: identifying the trigger requirement response for each trigger requirement condition; sending a trigger-specific alert for each trigger requirement condition to the patient device providing information about each identified trigger requirement condition and the trigger requirement response to be implemented; receiving an individual patient approval or rejection through the patient device for each trigger requirement response; and implementing the trigger responses that received the individual patient approval.

Additionally or alternatively, observation for the first trigger window condition and the second trigger window condition further comprises: detecting that the first trigger window condition and the second trigger window condition occur in a sequence within the same trigger window, wherein the neuromodulation therapy changes are implemented when the sequential trigger window conditions occur in a specific predefined order.

Additionally or alternatively, observation for the first trigger requirement condition and the second trigger requirement condition further comprises: detecting that the first trigger requirement condition and the second trigger requirement condition occur in a sequence within the same trigger window, wherein the neuromodulation therapy changes are implemented when the sequential trigger requirement conditions occur in a specific predefined order.

Additionally or alternatively, observation of the first trigger window condition and the second trigger window condition comprises receiving one or more user inputs through a patient device, each user input corresponding to the first trigger window condition or the second trigger window condition.

Additionally or alternatively, observation of the first trigger requirement condition and the second trigger requirement condition comprises receiving one or more user inputs through a patient device, each user input corresponding to the first trigger requirement condition or the second trigger requirement condition.

Additionally or alternatively, the received user input confirms at least one trigger window condition occurred. Additionally or alternatively, the received user input confirms at least one trigger window condition has not occurred.

Additionally or alternatively, upon reaching the trigger window end time in the absence of an observation of the trigger window trigger, changing the neuromodulation therapy delivered to the patient. Additionally or alternatively, in response to reaching the trigger window end time, changing the neuromodulation therapy delivered to the patient. Additionally or alternatively, in response to reaching the trigger requirement end time, preserving the neuromodulation therapy delivered to the patient.

Additionally or alternatively, after reaching the trigger requirement start time: initiating observation for an indication the trigger requirement trigger is not actively occurring. Additionally or alternatively, observation of the indication the trigger requirement trigger is not actively occurring initiates a hold off period. Additionally or alternatively, during the hold off period observation for the trigger requirement trigger is paused. Additionally or alternatively, observation of the trigger window trigger is a received user input. Additionally or alternatively, the received user input confirms an expected event occurred. Additionally or alternatively, the received user input confirms an expected event has not occurred. Additionally or alternatively, the trigger requirement trigger occurs for a predetermined length of time prior to changing the neuromodulation therapy delivered to the patient. Additionally or alternatively, the trigger requirement trigger is a lack of activity in the patient. Additionally or alternatively, the trigger window and the trigger requirement are spaced by a period of time during which changes to the neuromodulation therapy are not dependent on a trigger window trigger or trigger requirement trigger.

Another illustrative and non-limiting example takes the form of a method of delivering a neuromodulation therapy comprising: setting, for a neuromodulation system implanted in a patient, at least one trigger window, the at least one trigger window including a trigger window start time, a trigger window end time, and a definition of a trigger window trigger, and upon reaching the trigger window start time: initiating observation for the trigger window trigger; in response to observation of the trigger window trigger, changing a neuromodulation therapy delivered to the patient.

Additionally or alternatively, the definition of a trigger window trigger includes a first trigger window condition and a first trigger response, and a second trigger window condition and a second trigger response; the step of initiating observation for the trigger window trigger includes observing for each of the first trigger window condition and the second trigger window condition; and the step of changing a neuromodulation therapy delivered to the patient comprises either: in response to observing the first trigger window condition, executing the first trigger response to change the neuromodulation therapy; or in response to observing the second trigger window condition, executing the second trigger response to change the neuromodulation therapy.

Additionally or alternatively, each of the first trigger window condition and the second trigger window condition is at least one of: a user input received through a patient device; a sensed signal from an internal sensor of the neuromodulation system; or a sensed signal from an external sensor; and the first trigger response and the second trigger response are each: an amplitude change; a frequency change; a pulse width change; a fractionalization change; a program change; or a combination thereof.

Additionally or alternatively, changing the neuromodulation therapy further comprises: prior to implementing the first trigger response or the second trigger response, sending a notification to a patient device that the first trigger window condition or the second trigger window condition has been detected; and implementing the neuromodulation therapy change upon receiving a patient approval on the patient device.

Additionally or alternatively, changing the neuromodulation therapy comprises in response to not receiving a patient approval through the patient device, maintaining the current stimulation parameters.

Additionally or alternatively, the step of changing the neuromodulation therapy further comprises: detecting the first trigger window condition and the second trigger window condition during the same trigger window; for each detected trigger window condition: identifying the trigger response for each trigger window condition; sending a trigger-specific alert for each trigger window condition to the patient device providing information about each identified trigger window condition and the trigger response to be implemented; receiving an individual patient approval or rejection through the patient device for each trigger response; and implementing the trigger responses that received the individual patient approval.

Additionally or alternatively, observation for the first trigger window condition and the second trigger window condition further comprises: detecting that the first trigger window condition and the second trigger window condition occur in a sequence within the same trigger window, wherein the neuromodulation therapy changes are implemented when the sequential trigger window conditions occur in a specific predefined order.

Additionally or alternatively, observation of the first trigger window condition and the second trigger window condition comprises receiving one or more user inputs through a patient device, each user input corresponding to the first trigger window condition or the second trigger window condition.

Additionally or alternatively, the received user input confirms at least one trigger window condition occurred. Additionally or alternatively, the received user input confirms at least one trigger window condition has not occurred. Additionally or alternatively, upon reaching the trigger window end time in the absence of an observation of the trigger window trigger, changing the neuromodulation therapy delivered to the patient.

Additionally or alternatively, the method includes, in response to reaching the trigger window end time, changing the neuromodulation therapy delivered to the patient.

Additionally or alternatively, the method includes, in response to reaching the trigger requirement end time, preserving the neuromodulation therapy delivered to the patient.

Additionally or alternatively, after reaching the trigger requirement start time: initiating observation for an indication the trigger requirement trigger is not actively occurring.

Additionally or alternatively, observation of the indication the trigger requirement trigger is not actively occurring initiates a hold off period.

Additionally or alternatively, during the hold off period observation for the trigger requirement trigger is paused.

Additionally or alternatively, the trigger requirement trigger occurs for a predetermined length of time prior to changing the neuromodulation therapy delivered to the patient.

Additionally or alternatively, the trigger requirement trigger is a lack of activity in the patient.

Additionally or alternatively, the trigger window and the trigger requirement are spaced by a period of time during which changes to the neuromodulation therapy are not dependent on a trigger window trigger or trigger requirement trigger.

Another illustrative and non-limiting example takes the form of a method of delivering a neuromodulation therapy comprising: setting, for a neuromodulation system implanted in a patient, a trigger window and a trigger requirement: the trigger window including a trigger window start time, a trigger window end time, and a definition of a trigger window trigger, and the trigger requirement including a trigger requirement start time, a trigger requirement end time, and a definition of a trigger requirement trigger; upon reaching the trigger window start time: initiating observation for the trigger window trigger; in response to observation of the trigger window trigger, changing a neuromodulation therapy delivered to the patient; in response to reaching the trigger window end time, changing the neuromodulation therapy delivered to the patient; and upon reaching the trigger requirement start time: initiating observation for the trigger requirement trigger; in response to observation of the trigger requirement trigger, changing the neuromodulation therapy delivered to the patient; and in response to reaching the trigger requirement end time, preserving the neuromodulation therapy delivered to the patient.

Additionally or alternatively, the definition of a trigger window trigger includes a definition of a first trigger window condition and a first trigger response, and a definition of a second trigger window condition and a second trigger response; the step of initiating observation for the trigger window trigger includes observing for each of the first trigger window condition and the second trigger window condition; the step of changing a neuromodulation therapy delivered to the patient in response to observation of the trigger window trigger comprises either: in response to observing the first trigger window condition, executing the first trigger response to change the neuromodulation therapy; or in response to observing the second trigger window condition, executing the second window trigger response to change the neuromodulation therapy; the definition of a trigger requirement trigger includes a definition of a first trigger requirement condition and a first trigger requirement response, and a definition of a second trigger requirement condition and a second trigger requirement response; the step of initiating observation for the trigger requirement trigger includes observing for each of the first trigger requirement condition and the second trigger requirement condition; and the step of changing the neuromodulation delivered to the patient therapy in response to observation of the trigger requirement trigger comprises either: in response to observing the first trigger requirement condition, executing the first trigger requirement response to change the neuromodulation therapy; or in response to observing the second trigger requirement condition, executing the second trigger requirement response to change the neuromodulation therapy.

This overview is intended to provide an introduction to the subject matter of the present patent application. It is not intended to provide an exclusive or exhaustive explanation. The detailed description is included to provide further information about the present patent application.

### BRIEF DESCRIPTION OF THE DRAWINGS

In the drawings, which are not necessarily drawn to scale, like numerals may describe similar components in different views. Like numerals having different letter suffixes may represent different instances of similar components. The drawings illustrate generally, by way of example, but not by way of limitation, various embodiments discussed in the present document.

FIG. 1 shows a deep brain stimulation (DBS) system;

FIG. 2 shows an illustrative implantable pulse generator (IPG);

FIG. 3 shows a spinal cord stimulation (SCS) system;

FIG. 4 shows an illustrative flowchart of a method of delivering a neuromodulation therapy which accounts for variability in the user's schedule or routine;

FIG. 5 shows a schematic view of an illustrative program schedule that is adjusted based on one or more trigger events; and

FIG. 6 shows a schematic view of an illustrative program schedule that is adjusted based on one or more trigger events.

### DETAILED DESCRIPTION

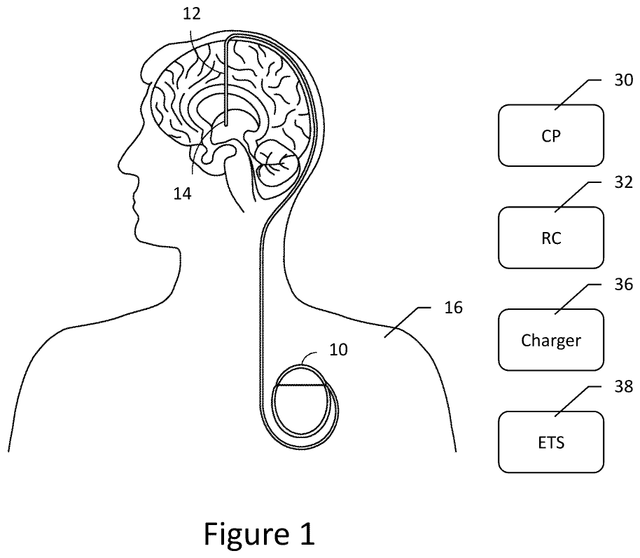

Implantable and/or wearable stimulations systems for the treatment of various diseases and disorders of the neurological system have proven effective in a wide variety of ways. For example, spinal cord stimulation (SCS) systems are accepted treatments for chronic pain syndromes while deep brain stimulation (DBS) systems may be used for the treatment of dementia, Alzheimer's disease, Parkinson's disease, dyskinesias, tremors, depression, anxiety or other mood disorders, sleep related conditions, etc. An illustrative DBS system and an illustrative SCS system are shown in FIGS. 1-3 and described in further detail below. An SCS or DBS system typically includes an Implantable Pulse Generator (IPG) 10. The IPG is coupled to tissue-stimulating electrodes carried on the distal end of one or more electrode leads that are implanted near the spinal column. The proximal ends of the one or more leads are tunneled through the patient's tissue to a location where the IPG is implanted. The proximal ends of the leads are coupled to the IPG to provide electrical stimulation from the electrodes to provide therapeutic benefits. Further details of FIGS. 1-3 are provided below.

In some cases, the IPG 10 may be configured to deliver neuromodulation therapy in accordance with one or more predefined stimulation programs. The predefined stimulation programs may be scheduled to delivery therapy at varying levels (intensities) at varying times of the day. The stimulation programs may operate on a schedule that is irrespective of the patient's local time or may occur in a time bound manner to sync with expected events such as, but not limited to, sleep or scheduled medications. However, patients may travel outside of their home time zones, sleep at different hours, or take their medications at varying times of the day. Thus, an ideal time or event-based stimulation schedule may be difficult to obtain as an estimate of fixed time points (e.g., sleep, medication, or the like) are inaccurate even for a same patient due to common life circumstances.

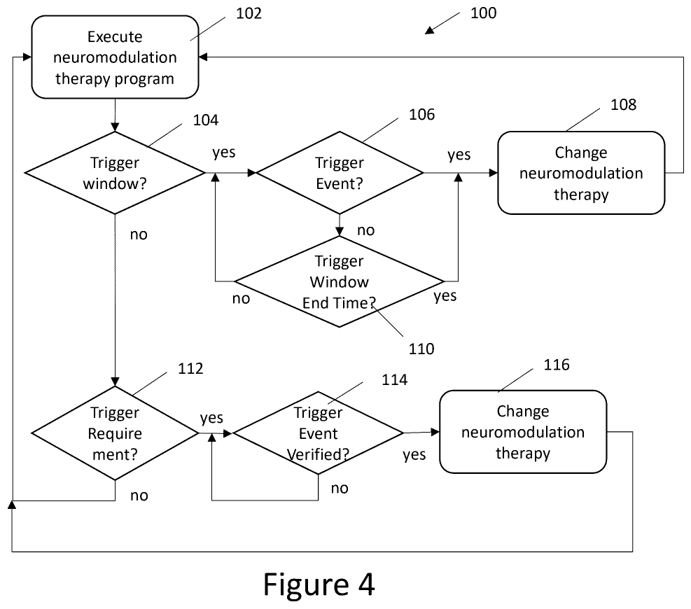

While time-based stimulation schedules may be meant to align to repeatable events such as sleeping and scheduled medication, not all users adhere to a strict schedule. For these users, scheduled program changes can be built in such that the IPG 10 waits to begin a change in the modulation therapy until either the IPG 10 has confirmed that the event has occurred, confirmed that the event has not occurred, or that a set period of time has elapsed. FIG. 4 is an illustrative method 100 of delivering a neuromodulation therapy which accounts for variability in the user's schedule or routine. Prior to implanting the IPG 10 or during the therapy configuration and testing phase, a stimulation schedule or neuromodulation therapy schedule/program may be determined and programmed into the IPG 10. In some cases, the stimulation schedule may be altered or changed after implantation of the IPG 10.

The neuromodulation therapy schedule may include one or more preset changes to the neuromodulation therapy that is delivered. In some cases, one or more triggers or trigger events may be used to initiate the changes to the delivered neuromodulation therapy. This may allow the neuromodulation therapy to be adapted to change based on triggers or trigger events related to the actions of the patient instead of relying only on time-based changes. Some illustrative triggers or trigger events may include taking a medication, sleep onset, waking onset, exercise or other activity, meals, etc. In some cases, a trigger event may initiate a change in the neuromodulation therapy in response to an action received at a connected device, a detected action, or in response to the expiration of a window of time for said trigger event to occur. In other examples, a trigger event may require verification that said trigger event has occurred prior to a change in neuromodulation therapy to occur. The trigger events may occur during one or more trigger windows or one or more trigger requirements. The trigger windows and/or trigger requirements may be set or programmed within the control program.

In an example, the IPG includes a motion sensor, and lack of patient activity may be detected by monitoring an output of the motion sensor, and such lack of activity can be a trigger. In an example, the IPG again includes a motion sensor, an onset or initiation of patient activity may be detected by monitoring an output of the motion sensor, and onset or initiation (or just patient activity in general) may serve as a trigger. The motion sensor may include, for example, an accelerometer, such as a multi-axis accelerometer. In an example, the IPG may include a communication circuit for communicating with the external devices, such as a patient device (a patient remote control for example) and/or other devices, such as a Bluetooth-enabled fitness or motion tracker worn by the patient on, for example, a smartwatch or other item. A communication from such an external device may indicate onset, initiation, or lack of patient activity, and the IPG may use onset, initiation or lack of patient activity as a trigger.

Once the neuromodulation therapy schedule has been loaded into the IPG 10, the IPG 10 may execute the program and deliver therapy to the patient in accordance with the schedule, as shown at block 102. The neuromodulation therapy schedule may be configured to look for trigger windows and trigger requirements which have been programmed into the control schedule during execution of the neuromodulation therapy program. Trigger windows may identify time periods during which a first type of trigger event is expected. During or after a trigger window, the IPG 10 may change the neuromodulation therapy delivered to the patient based on a confirmed or expected trigger event. Trigger requirements may identify time periods during which a second type of trigger event, different from the first type of trigger event, is expected. During the trigger requirement, the IPG 10 may only change the neuromodulation therapy delivered if the second type of trigger event is confirmed to have occurred or is actively occurring.

As the neuromodulation therapy program is executing, the IPG 10 may be configured to look for trigger windows that are programmed in the control program, as shown at block 104. The IPG 10 may include one or more trigger windows which each include a trigger window start time, a trigger window end time, and a definition of the trigger window trigger or trigger event, where each trigger event may correspond to either a first trigger window condition or a second trigger window condition, each associated with its respective first or second trigger response.

Upon reaching the trigger window start time, the trigger window is detected and the IPG 10 may initiate observation for the trigger window triggers or trigger events. The trigger window start time may correspond to the earliest time point in a time window during which a trigger event is expected. For example, if a patient is taking a medication every four to six hours while awake, the trigger window start time may correspond to four hours from the time of the last dose or last expected dose. In other examples, the trigger window start time may be at predefined times that are not dependent on a previous trigger event. It is contemplated that each trigger window may include one or more trigger window triggers or trigger events. In one illustrative example, a trigger window trigger or trigger event may be an expected dose of medication. For example, the patient may be prescribed a medication that is to be taken at predetermined intervals throughout the day while the patient is awake.

The IPG 10 may be programmed with one or more trigger windows which each define or correspond to a window or period of time during which the patient is expected to take the medication. While the trigger window(s) is described with respect to an expected dose of medication, the trigger window(s) may have additional or alternative trigger events. During the trigger window (e.g., between the trigger window start time and the trigger window end time), the IPG 10 may detect or observe the trigger event specific to that particular trigger window, as shown at block 106, which may include detecting the first trigger window condition and the second trigger window condition. In response to observation of either the first trigger window condition or the second trigger condition, the IPG 10 may be configured to execute either the first trigger response or the second trigger response, respectively, to change a neuromodulation therapy delivered to the patient. For example, the IPG 10 may be programmed to change a parameter of the stimulation in response to the trigger event that is input or detected, as shown at block 108.

The IPG 10 may then continue to execute the neuromodulation therapy program as programmed with the detected/observed trigger window trigger or trigger event being the variable which initiates a therapy change. Some illustrative parameters that may be changed include, but are not limited to, amplitude, frequency, pulse width, turning stimulation on/off, initiating a new program, or the like. In some embodiments, the IPG 10 may require the first and second trigger window conditions to occur in a specific predefined sequence within the same trigger window before implementing the therapy change.

In one illustrative example, when it is time for a patient to take their medication, an alert may be displayed at a user remote control (RC) 32, or other user device configured to communication with the IPG 10. The alert may be transmitted to the user at the trigger window start time. However, the alert may be transmitted at any time during the trigger window. The RC 32 may be configured to receive a user input in response to the alert. For example, the user input may confirm the medication was taken or may allow the user to indicate that the medication was not taken. The IPG 10 may adjust the neuromodulation therapy according the user input. For example, if the user indicates they have taken their medication, the IPG 10 may adjust the therapy according a predetermined plan. In some cases, the predetermined plan may include a time delay to allow the medication time to take effect prior to changing the neuromodulation therapy. If the user actively indicates that they have not taken their medication, the IPG 10 may be configured to adjust the therapy according to a different predetermined plan or may continue with a current therapy.

In some embodiments, the IPG 10 may not receive confirmation that the user did or did not take the medication. If a user input is not received, the IPG 10 may look for the trigger window end time, as shown at block 110. In some cases, the IPG 10 may maintain a current treatment until the trigger window end time. Upon reaching the trigger window end time, the IPG 10 may change the neuromodulation therapy delivered to the patient. In one example, at the trigger window end time, the IPG 10 may initiate a predetermined plan that assumes the patient has taken the medication, as shown at block 108. However, this is not required. For example, in some cases, the IPG 10 may initiate a predetermined plan that assumes the patient has not taken the medication. In some cases, the IPG 10 may be configured to maintain a count of how often or with what frequency a trigger event goes unacknowledged by the patient. In some cases, the number of trigger events which are not confirmed may determine how the IPG 10 changes the neuromodulation therapy. If a predetermined number of triggers are missed over a predetermined length of time, the IPG 10 may be configured to send an alert to a caregiver. Alternatively, or additionally, the IPG 10 may be configured to implement an alternative neuromodulation therapy schedule if a predetermined number of triggers are missed over a predetermined length of time.

As described above, in some embodiments, the IPG 10 may not change a neuromodulation therapy until a trigger has been met or verified. As the neuromodulation therapy program is executing, the IPG 10 may be configured to look for trigger requirements that are programmed in the control program, as shown at block 112. The IPG 10 may include one or more trigger requirements which each include a trigger requirement start time, a trigger requirement end time, and a definition of the trigger requirement trigger or trigger event. In some examples, each trigger requirement trigger or trigger event may correspond to either a first trigger requirement condition or a second trigger requirement condition, each associated with its respective first trigger requirement response or second trigger requirement response. In response to reaching the trigger requirement start time, the IPG 10 may initiate observation for the trigger requirement trigger or trigger event, as shown at block 114, which may include detecting the first trigger requirement condition and the second trigger requirement condition. If the first trigger requirement condition or the second requirement condition is positively verified or observed, the IPG 10 may be configured to execute either the first trigger requirement response or the second trigger requirement response, respectively, to change the neuromodulation therapy delivered to the patient. For example, the IPG 10 may initiate a predetermined plan in accordance with the activity associated with the trigger requirement trigger, as shown at block 116. In some embodiments, the IPG 10 may require the first and second trigger requirement conditions to occur in a specific predefined sequence within the same trigger window before implementing the therapy change.

In one illustrative example, the IPG 10 may be configured to verify a patient is asleep before changing the neuromodulation therapy. The IPG 10 may be configured to look for extending periods of time of a lack of activity of the patient. A lack of activity may be detected by a lack of movement of the patient and/or no detectable activity associated with an awake state on any connected device for a set window of time. In some cases, the extended period of time may be about 30 minutes or greater. However, the extended period of time may be less than 30 minutes or greater than 30 minutes, as desired. In some embodiments, the IPG 10 may be configured to start looking for a lack of activity at a predetermined time corresponding to the trigger requirement start time. In some cases, the predetermined time may be around a time a user typically falls asleep. In other examples, the predetermined time may be a predetermined length of time before a time a user typically falls asleep. In yet other examples, the IPG 10 may be configured to look for a lack of activity at predetermined intervals throughout the day. It is contemplated that if activity (e.g., indicating that the patient is awake) is detected after the trigger requirement start time, the IPG 10 may be configured to wait for a predetermined length of time or hold off period prior to restarting the inspection for lack of activity. The hold off period may be about 15 minutes or greater. However, the hold off period may be less than 15 minutes or greater than 15 minutes, as desired. Said differently, a hold off period may occur if the IPG 10 observes the trigger requirement trigger is not occurring. Once the neuromodulation therapy has been changed, the IPG 10 may be configured to control the delivery of the therapy in accordance with the control program. In response to reaching the trigger requirement end time, the current neuromodulation therapy may be preserved. For example, if the neuromodulation therapy has been changed due to observation of a trigger requirement trigger or if the neuromodulation therapy has not been changed due to lack of observation of the trigger requirement trigger, the neuromodulation therapy that is being delivered at the trigger requirement end time is preserved or maintained.

It is contemplated that there may be time periods between adjacent trigger windows, between adjacent trigger requirements, and/or between adjacent trigger windows and trigger requirements. These times between trigger windows and/or trigger requirements may be a “stable” time or stable mode. During the stable mode, the neuromodulation therapy may not be dependent on information received from one or more connected devices. During stable mode, the IPG 10 may execute the programmed neuromodulation therapy pattern until the chronologically next trigger window or trigger requirement.

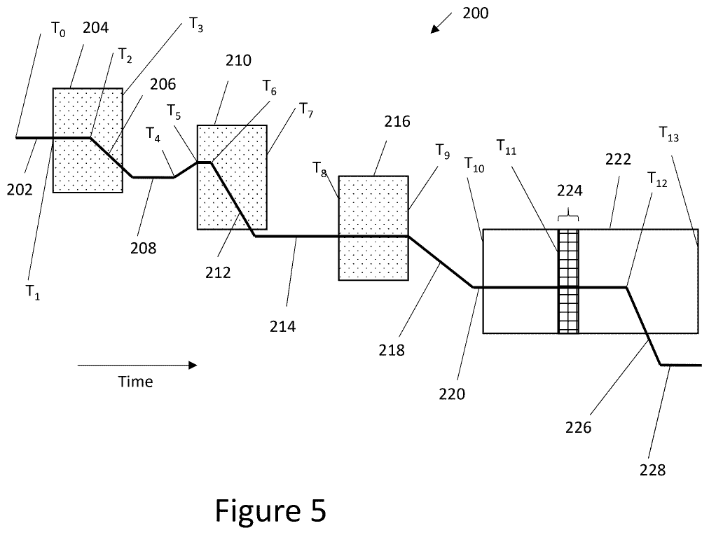

FIG. 5 is a schematic view of an illustrative program schedule 200 that is adjusted based on one or more trigger events. In the illustrative example, the program schedule is shown beginning at a first time T₀. The neuromodulation therapy is schematically illustrated as line 202. It should be understood that the neuromodulation therapy program runs continuously and T₀ is merely illustrative and may represent any time during a 24-hour time period. At T₀, the IPG 10 is operating in a stable time mode. At time T₁, a trigger window time of a first trigger window 204 occurs. During the first trigger window 204, the IPG 10 may look for confirmation of a trigger event. As described above, the confirmation of the trigger event may be received at a connected device, such as, but not limited to an RC 32. At T₂, the IPG 10 has received a confirmation that the trigger event has occurred. For example, the IPG 10 may receive confirmation that the patient took an expected dose of medication. Upon confirmation of the trigger event, the IPG 10 may adjust the neuromodulation therapy 202 according a predetermined program that accounts for the effects of the trigger event. In the illustrated example, the neuromodulation therapy gradually changes (shown at 206) from the therapy at time T₂ to a new stable therapy shown at 208. However, this is not required. In some cases, the neuromodulation therapy 202 may undergo an abrupt or step-wise change upon confirmation of the trigger event. In other cases, the neuromodulation therapy 202 may be held steady for a predetermined length of time after the confirmation of the trigger event and then adjusted in a gradual or stepwise manner. At time T₃, the trigger window end time occurs. However, as the neuromodulation therapy 202 has already been changed in response to the confirmation of the trigger event, T₃ passes with no additional changes to the neuromodulation therapy 202.

The neuromodulation therapy 202 may operate in a stable mode 208 for a period of time. At time T₄, the IPG 10 may be configured to anticipate a change in state of the patient. For example, a medication may be expected to become less effective and the neuromodulation therapy 202 adjusted accordingly. However, this is not required and the neuromodulation therapy 202 may remain unchanged. At time T₅, a trigger window time of a second trigger window 210 occurs. During the second trigger window 210, the IPG 10 may look for confirmation of a trigger event. As described above, the confirmation of the trigger event may be received at a connected device, such as, but not limited to an RC 32. At T₆, the IPG 10 has received a confirmation that the trigger event has occurred. Upon confirmation of the trigger event, the IPG 10 may adjust the neuromodulation therapy 202 according a predetermined program that accounts for the effects of the trigger event. In the illustrated example, the neuromodulation therapy gradually changes (shown at 212) from the therapy at time T₆ to a new stable therapy shown at 214. However, this is not required. In some cases, the neuromodulation therapy 202 may undergo an abrupt or step-wise change upon confirmation of the trigger event. In other cases, the neuromodulation therapy 202 may be held steady for a predetermined length of time after the confirmation of the trigger event and then adjusted in a gradual or stepwise manner. At time T₇, the trigger window end time occurs. However, as the neuromodulation therapy 202 has already been changed in response to the confirmation of the trigger event, T₇ passes with no additional changes to the neuromodulation therapy 202.

The neuromodulation therapy 202 may operate in a stable mode 214 for a period of time. At time T₈, a trigger window time of a third trigger window 216 occurs. During the third trigger window 216, the IPG 10 may look for confirmation of a trigger event. As described above, the confirmation of the trigger event may be received at a connected device, such as, but not limited to an RC 32. At time T₉, the trigger window end time occurs. In this example, the third trigger window 216 has elapsed with no confirmation of the trigger event. In the absence of confirmation of the trigger event, at the trigger window end time T₉, the IPG 10 may adjust the neuromodulation therapy 202 according a predetermined program that accounts for the effects of the trigger event. In the illustrated example, the neuromodulation therapy gradually changes (shown at 218) from the therapy at time T₉ to a new stable therapy shown at 220. However, this is not required. In some cases, the neuromodulation therapy 202 may undergo an abrupt or step-wise change upon confirmation of the trigger event. In other cases, the neuromodulation therapy 202 may be held steady for a predetermined length of time after the confirmation of the trigger event and then adjusted in a gradual or stepwise manner.

It is contemplated that the trigger events for each of the first, second, and third trigger windows 204, 210, 216 may be the same or different. Further, the neuromodulation therapy program may include fewer than three or more than three trigger windows, as desired.

The neuromodulation therapy 202 may operate in a stable mode 220 for a period of time. At time T₁₀, a trigger requirement start time of a trigger requirement 222 occurs. During the trigger requirement 222, the IPG 10 may look for confirmation of a trigger requirement event. In the illustrated example, the trigger requirement event may be the patient is sleeping. this is just an example and not intended to be limiting. As described above, the confirmation of the trigger requirement event may be the absence of any detectable activity associated with an awake state on any connected device for a set window of time. At time T₁₁, the IPG 10 detects activity associated with an awake state. For example, the patient may be actively using a connected device. When the activity associated with an awake state is detected, the IPG 10 may initiate a hold off time period 224 during which the IPG 10 does not attempt to actively confirm the trigger requirement event. At the expiration of the hold off time period, the IPG 10 may again look for confirmation of the trigger requirement event. At time T₁₂, the IPG 10 has determined that the trigger requirement event has occurred. Upon confirmation of the trigger requirement event, the IPG 10 may adjust the neuromodulation therapy 202 according a predetermined program that accounts for the effects of the trigger requirement event. In the illustrated example, the neuromodulation therapy gradually changes (shown at 226) from the therapy at time T₁₂ to a new stable therapy shown at 228. However, this is not required. In some cases, the neuromodulation therapy 202 may undergo an abrupt or step-wise change upon confirmation of the trigger event. In other cases, the neuromodulation therapy 202 may be held steady for a predetermined length of time after the confirmation of the trigger event and then adjusted in a gradual or stepwise manner. At time T₁₃, the trigger requirement end time occurs. However, as the neuromodulation therapy 202 has already been changed in response to the confirmation of the trigger event, T₁₃ passes with no additional changes to the neuromodulation therapy 202. In some embodiments, the IPG 10 may require confirmation that the trigger requirement event has occurred for a predetermined length of time before the neuromodulation therapy 202 is adjusted.

It is contemplated that the trigger requirement event for the trigger requirement 222 may be different that the trigger events for one or more of the first, second, and third trigger windows 204, 210, 216. However, this is not required. Further, the neuromodulation therapy program may include fewer than one or more than one trigger requirements, as desired.

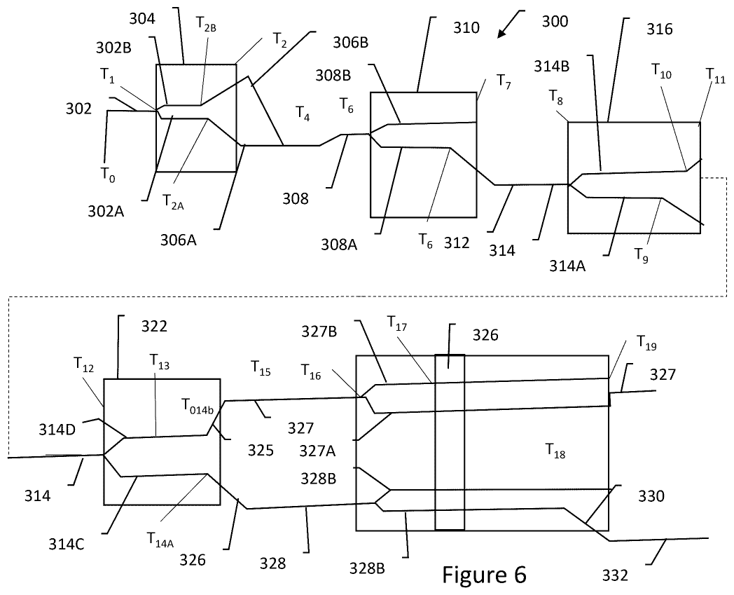

FIG. 6 is a schematic view of an illustrative program schedule 300 that is adjusted based on one or more trigger events, showing a schedule where one or more trigger conditions may be detected and responded to within the same trigger window. More specifically, the IPG 10 is monitoring for both the first trigger window condition and the second trigger window condition.

In the illustrative example, the program schedule is shown beginning at a first time T₀. The neuromodulation therapy is schematically illustrated as line 302. It should be understood that the neuromodulation therapy program runs continuously and T₀ is merely illustrative and may represent any time during a 24-hour time period. At T₀, the IPG 10 is operating in a stable time mode.

During the first trigger window 304, the IPG 10 may look for confirmation of at least one trigger event. As described above, the confirmation of the trigger event may be received at a connected device, such as, but not limited to an RC 32. At time T₁, a trigger window time of a first trigger window 304 occurs, line 302 splitting into two branches, 302a, 302b, representing the potential therapy paths in response to fulfillment of the first and second trigger window conditions, respectively. At T_{2A} and T_{2B}, the IPG 10 has received a confirmation that both the first trigger window condition 302a and the second trigger window condition 302b have been met in response to at least one trigger event. For example, the IPG 10 may receive confirmation that the patient took an expected dose of medication.

Upon confirmation of the trigger events, the IPG 10 may adjust the neuromodulation therapy 302 according a predetermined program that accounts for the effects of each trigger event. In the illustrated example, the neuromodulation therapy gradually changes (shown at 306a and 306b) from the therapy at time T_{2A,2B} and converges to a new stable therapy shown at 308. However, this is not required and may not result from the changes and triggers. In some cases, the neuromodulation therapy 302 may undergo an abrupt or step-wise change upon confirmation of the trigger event. In other cases, the neuromodulation therapy 302 may be held steady for a predetermined length of time after the confirmation of the trigger event and then adjusted in a gradual or stepwise manner. At time T₃, the trigger window end time occurs. However, as the neuromodulation therapy 302 has already been changed in response to the confirmation of the trigger event, T₃ passes with no additional changes to the neuromodulation therapy 302.

The neuromodulation therapy 302 may operate in a stable mode 308 for a period of time. At time T₄, the IPG 10 may be configured to anticipate a change in state of the patient. For example, a medication may be expected to become less effective and the neuromodulation therapy 302 adjusted accordingly. However, this is not required and the neuromodulation therapy 302 may remain unchanged. At time T₅, a trigger window time of a second trigger window 310 occurs. During the second trigger window 310, the IPG 10 may look for confirmation of a trigger event that fulfills the first trigger window condition and/or the second trigger window condition (shown at branches 308a, 308b). As described above, the confirmation of the trigger event may be received at a connected device, such as, but not limited to an RC 32. At T₆, the IPG 10 has received a confirmation that the first trigger window condition has been met in response to a trigger event. In turn, first trigger response is executed. In the illustrated example, the neuromodulation therapy gradually changes (shown at 312) from the therapy at time T₆ to a new stable therapy shown at 314. However, this is not required. In some cases, the neuromodulation therapy 302 may undergo an abrupt or step-wise change upon confirmation of the trigger event. In other cases, the neuromodulation therapy 302 may be held steady for a predetermined length of time after the confirmation of the trigger event and then adjusted in a gradual or stepwise manner. At time T₇, the trigger window end time occurs. Throughout the second trigger window 310, The IPG 10 did not detect the occurrence of a trigger event that would fulfill the second trigger window condition, thus branch 308b terminates at the end of the second trigger window 310 at T₇. However, as the neuromodulation therapy 302 has already been changed in response to fulfillment of the first trigger window condition, T₇ passes with no additional changes to the neuromodulation therapy 302.

The neuromodulation therapy 202 may operate in a stable mode 314 for a period of time. At time T₈, a trigger window time of a third trigger window 316 occurs. During the third trigger window 316, the IPG 10 may look for confirmation of a trigger event that fulfills the first trigger window condition and/or the second trigger window condition (shown at branches 314a, 314b). As described above, the confirmation of the trigger event may be received at a connected device, such as, but not limited to an RC 32. In some examples, the IPG 10 may adjust the neuromodulation therapy 302 according to a predetermined program that requires the first and second trigger window conditions to be met in a specific predefined sequence before implementing therapy changes.

For example, the patient/user may take a medication, treated by the system as a first trigger, but the system may wait to change therapy until evidence of the medication taking effect, wherein the evidence of the medication taking effect is treated as the second trigger. Evidence of the medication taking effect may be an anticipated response changing a physiological signal, a symptom, or even an overall change in activity level. Examples may include such as cessation of a tremor, relaxation of a muscle, change in heart rate, or any other response to medication that may be sensed by the system. The first and second triggers can be described as “stacked” triggers, or related triggers.

Another example may be the patient eating a meal, which can be a first trigger, and then taking a medication, which can be the second trigger. The time that passes between the first and second triggers may be used to adjust the time between the second trigger and the time at which therapy changes. For example, if a medication is taken immediately after a meal is eaten, the time to the medication taking effect may be different than if the medication is taken a longer period of time after a meal. Further, in some instances, the type of meal consumed can affect the delay from second trigger to therapy start, if, for example, the type of food affects the response rate of the patient to the medication.

In the illustrated example, the second trigger window condition is required to be met prior to the first trigger window condition. At time T₉, the IPG 10 has received a confirmation that the first trigger window condition has been met in response to a trigger event. At time T₁₀, the IPG 10 has received a confirmation that the second trigger window condition has been met in response to the trigger event. At time T₁₁, the trigger window end time occurs. Because the second trigger window condition was not met prior to the first trigger window condition as was predefined, neither the first trigger response nor the second trigger response are executed. Consequently, in the illustrated example, the neuromodulation therapy 302 remains unchanged at the close of the third trigger window, and operates in the stable mode 314 for a period of time. At time T₁₂, a trigger window time of a fourth trigger window 322 occurs, the fourth trigger window 322 having the same predetermined program that requires the second trigger window condition to be met prior to the first trigger window condition. During the third trigger window 316, the IPG 10 may look for confirmation of a trigger event that fulfills the first trigger window condition and/or the second trigger window condition (shown at branches 314c, 314d). As described above, the confirmation of the trigger event may be received at a connected device, such as, but not limited to an RC 32. At time T₁₃, the IPG 10 has received a confirmation that the second trigger window condition has been met, however no response is yet executed.

At T_{14A}, the IPG 10 has received a confirmation that the first trigger window condition has been met. Because the second trigger window condition was met prior to the first trigger widow condition as predefined, both the first trigger response and the second trigger response are executed at T_{14A,14B}. Upon confirmation of the trigger event that fulfills the trigger window conditions in the predefined sequence, the IPG 10 may adjust the neuromodulation therapy 302 according the predetermined program that accounts for the effects of the trigger event. In the illustrated example, the neuromodulation therapy gradually changes (shown at 325, 326) from the therapy at time T_{14A,14B} to two new stable therapies shown at 327, 328.

The new therapies 327, 238 may represent a distinct therapy configuration. For example, the first trigger window condition may lead to execution of a first trigger response that changes one set of therapy parameters (such as amplitude) and the second trigger window condition may lead to execution of a second trigger responses that changes a different set of therapy parameters (such as frequency). Gradual change into the new therapies 327, 328 is not required. In some cases, the neuromodulation therapy 302 may undergo an abrupt or step-wise change upon confirmation of the trigger event. In other cases, the neuromodulation therapy 302 may be held steady for a predetermined length of time after the confirmation of the trigger event and then adjusted in a gradual or stepwise manner. At time T₁₅, the trigger window end time occurs. However, as the neuromodulation therapy 302 has already been changed in response to the confirmation of the trigger event, T₁₅ passes with no additional changes to the neuromodulation therapy 302. Accordingly, the resulting therapies 327 and 328 may remain independent therapy paths beyond the trigger window end time T₁₅.

It is contemplated that the first and second trigger window conditions for each trigger window may be the same or different, and their corresponding responses may be customized based on which conditions are detected. Further, the neuromodulation therapy program may include multiple trigger windows and requirements, each capable of responding to multiple trigger conditions, as desired.

Additionally, it is contemplated that the trigger events for each of the first, second, and third trigger windows 304, 310, 316, 322 may be the same or different. Further, the neuromodulation therapy program may include fewer than four or more than four trigger windows, as desired.

The neuromodulation therapy 302 may operate in stable modes 328, 327 for a period of time. At time T₁₆, a trigger requirement start time of a trigger requirement 324 occurs. During the trigger requirement 324, the IPG 10 may look for confirmation of a trigger requirement event that fulfills at least one trigger requirement trigger condition (shown as branches 327a, 327b, 328a, 328b. In the illustrated example, the trigger requirement event may be the patient is sleeping. this is just an example and not intended to be limiting. As described above, the confirmation of the trigger requirement event may be the absence of any detectable activity associated with an awake state on any connected device for a set window of time.

At time T₁₇, the IPG 10 detects activity associated with an awake state. For example, the patient may be actively using a connected device. When the activity associated with an awake state is detected, the IPG 10 may initiate a hold off time period 326 during which the IPG 10 does not attempt to actively confirm the trigger requirement event. At the expiration of the hold off time period, the IPG 10 may again look for confirmation of the trigger requirement event. At time T₁₈, the IPG 10 has determined that the trigger requirement event that meets the trigger requirement condition of branch 328a, has occurred. Upon confirmation of the trigger requirement event, the IPG 10 may adjust the neuromodulation therapy 302 according a predetermined program that accounts for the effects of the trigger requirement event. In the illustrated example, the neuromodulation therapy gradually changes (shown at 330) from the therapy at time Tis to a new stable therapy shown at 332. However, this is not required. In some cases, the neuromodulation therapy 302 may undergo an abrupt or step-wise change upon confirmation of the trigger event. In other cases, the neuromodulation therapy 302 may be held steady for a predetermined length of time after the confirmation of the trigger event and then adjusted in a gradual or stepwise manner. At time T₁₉, the trigger requirement end time occurs. However, as the neuromodulation therapy 302 has already been changed in response to the confirmation of the trigger event, T₁₉ passes with no additional changes to the neuromodulation therapy 302. In some embodiments, the IPG 10 may require confirmation that the trigger requirement event has occurred for a predetermined length of time before the neuromodulation therapy 302 is adjusted.

It is contemplated that the trigger requirement event for the trigger requirement 324 may be different that the trigger events for one or more of the first, second, third, and fourth trigger windows 304, 310, 316, 322. However, this is not required. Further, the neuromodulation therapy program may include fewer than one or more than one trigger requirements, as desired.

FIGS. 1-3 will now be discussed in greater detail. FIG. 1 shows an illustrative deep brain stimulation (DBS) system implanted in a patient. The system comprises an implantable pulse generator (IPG) 10, shown implanted in the pectoral region of a patient 16. The IPG 10 is coupled to a lead 12 which extends subcutaneously to the head of the patient 16, through a burr hole formed in the patient's skull, and then into the brain. In the example shown, the lead 12 includes a plurality of electrodes positioned near the distal end 14 of the lead. The lead 12 may be placed at any suitable location of the brain where a target for therapy is identified. For example, a lead 12 may be positioned so that the distal end 14 is near the mid-brain and/or various structures therein that are known in the art for use in providing stimulation to treat various diseases. The lead may use ring electrodes or may use other known structures, including segmented or directional lead electrodes that do not fully encircle the lead body.

DBS may be targeted, for example, and without limitation, at neuronal tissue in the thalamus, the globus pallidus, the subthalamic nucleus, the pedunculopontine nucleus, substantia nigra pars reticulate, the cortex, the globus pallidus externus, the medial forebrain bundle, the periaquaductal gray, the periventricular gray, the habenula, the subgenual cingulate, the ventral intermediate nucleus, the anterior nucleus, other nuclei of the thalamus, the zona incerta, the ventral capsule, the ventral striatum, the nucleus accumbens, and/or white matter tracts connecting these and other structures. Data related to DBS may include the identification of neural tissue regions determined analytically to relate to side effects or benefits observed in practice. “Targets” as used herein are brain structures associated with therapeutic benefits, in contrast to avoidance regions or “Avoid” regions which are brain structures associated with side effects.

Conditions to be treated may include dementia, Alzheimer's disease, Parkinson's disease, dyskinesias, tremors, depression, anxiety or other mood disorders, sleep related conditions, etc. Therapeutic benefits may include, for example, and without limitation, improved cognition, alertness, and/or memory, enhanced mood or sleep, elimination, avoidance or reduction of pain or tremor, reduction in motor impairments, and/or preservation of existing function and/or cellular structures, such as preventing loss of tissue and/or cell death. Therapeutic benefits may be monitored using, for example, patient surveys, performance tests, and/or physical monitoring such as monitoring gait, tremor, etc. Side effects can include a wide range of issues such as, for example, and without limitation, reduced cognition, neuroinflammation, alertness, and/or memory, degraded sleep, depression, anxiety, unexplained weight gain/loss, tinnitus, pain, tremor, etc. These are just examples, and the discussion of ailments, benefits, and side effects is merely illustrative and not exhaustive.

The illustrative system of FIG. 1 includes various external devices. A clinician programmer (CP) 30 may be used to determine/select therapy programs, including steering, as well as stimulation parameters. The CP 30 can be used by a physician, or at the direction of a physician, to obtain data from and provide instructions to the IPG 10 via suitable communications protocols such as Bluetooth or MedRadio or other wireless communications standards, and/or via other modalities such as inductive telemetry. Stimulation parameters may include amplitude of stimulation pulses, frequency or repetition rate of stimulation pulses, pulse width of stimulation pulses, and more complex parameters such as burst definition, as are known in the art. Biphasic square waves are commonly used, though nothing in the present invention is limited to biphasic square waves, and ramped, triangular, sinusoidal, monophasic, and other stimulation types may be used as desired.

The CP 30 may be, for example and without limitation, a computer such as a laptop or tablet computer. The CP 30 therefore includes a microcontroller and/or microprocessor, and associated memory. The memory may take any suitable form (RAM, ROM, Flash, etc.), and stores machine readable instructions allowing the processor to perform the methods disclosed herein. To the extent Bluetooth is used as a communications protocol, the RF circuitry may be included in the device as a communications circuitry, located internal to the CP 30. If some other communications technology (inductive or Medradio) is used, or if range is limited by the IPG 10, for example, the communications circuit may be provided via a wand having specialized circuitry (for Bluetooth, Medradio, or inductive telemetry) therein that couples, for example, to a USB port on the CP 30. The CP 30 may include a user interface, such as a screen or touchscreen, keyboard, mouse, trackball, etc. allowing the user to provide instructions and make choices.

A patient remote control (RC) 32 can be used by the patient to perform various actions relative to the IPG 10. These may be physician defined options, and may include, for example, turning therapy on and/or off, entering requested information (such as answering questions about activities, therapy benefits, and side effects), and making (limited) adjustments to therapy such as selecting from available therapy programs and adjusting, for example, amplitude settings. The RC 32 can communicate via similar telemetry as the CP 30 to control and/or obtain data from the IPG 10. The patient RC 32 may also be programmable on its own, or may communicate or be linked with the CP 30. The RC 32 may be a dedicated device, including a custom device, a locked off-the-shelf device with specialized software to prevent other uses, or may be a multi-purpose device such as the patient's smart phone.

A charger 36 may be provided to the patient to allow the patient to recharge the IPG 10, if the IPG 10 is rechargeable. In some systems, the IPG 10 is not rechargeable, and so the charger 36 may be omitted. The charger 36 can operate, for example, by generating a varying magnetic field (such as via an inductor) to activate an inductor associated with the IPG 10 to provide power to recharge the IPG battery, using known methods and circuitry.

Some systems may include an external test stimulator (ETS) 38. The ETS 38 can be used to test therapy programs after the lead 12 has been implanted in the patient to determine whether therapy will or can work for the patient 16. For example, an initial implantation of the lead 12 can take place using, for example, a stereotactic guidance system, with the IPG 10 temporarily left out. After a period of healing, the patient may return to the clinic for therapy configuration and testing. The lead 12 may have a proximal end thereof connected to an intermediate connector (sometimes called an operating room cable) that couples to the ETS 38, and the ETS 38 can be programmed using the CP 30 with various therapy programs and stimulation parameters. Once therapy suitability for the patient is established to the satisfaction of the patient 16 and/or physician, the permanent IPG 10 is implanted and the lead 12 is connected thereto, with the ETS 38 then removed from use.

A vagal stimulation system may be provided located near the vagus nerve. This may be in place of the DBS IPG 10 and lead 12, if desired, or may be an additional stimulator for the patient 16. Stimulation devices may be microstimulators, and may include or exclude a lead, as desired. Some example microstimulators are can be observed in U.S. Pat. No. 8,127,424, the disclosure of which is incorporated herein by reference. Devices, including microstimulators, may be externally powered or internally powered, as desired.

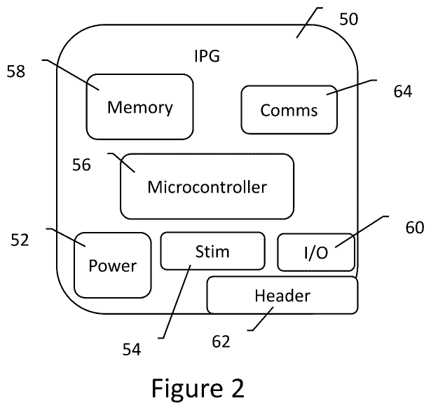

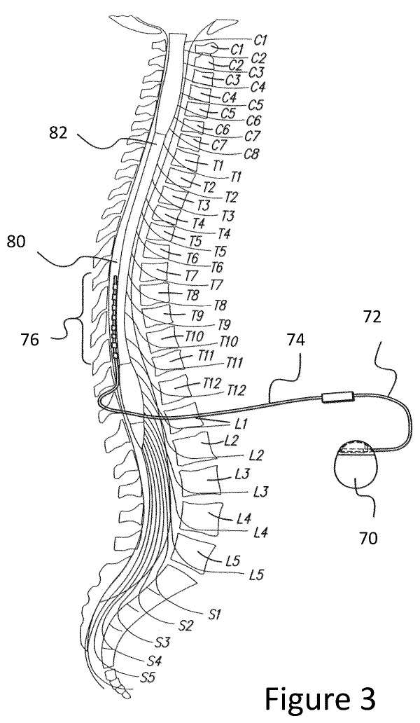

FIG. 2 shows an illustrative IPG 10 in block form. The IPG 10 may have a suitable hermetic housing 50, which may be conductive (titanium, stainless steel, etc.) in order to serve as an additional electrode in the system. Inside the housing 50 there is a power supply 52, which may include one or more batteries (rechargeable or not), along with charging circuitry (if rechargeable) and controlled voltage supplies (as desired and suitable to the system). Stimulation circuitry is shown at 54, and provides outputs for the system to use in therapy. A microcontroller 56 is also provided and may be associated with a memory 58. The microcontroller 56 may also be in the form of a microprocessor. Any suitable arrangement of additional systems and circuitry may be included, such as additional logic, communications bus, application specific integrated circuits, etc. The memory 58 may include any of RAM, ROM, and/or Flash memory, or other memory devices/media, and stores machine readable instructions for performing the methods disclosed herein and providing device configurations as described herein. The stimulation circuitry 54 issues therapy pulses, which are directed in accordance with input/output circuitry 60 (which may include a plurality of switches to allow selection of electrodes for use in outputs), that directs signals to and receives signals from a connector block in the header 62. The connector block in the header 62 is part of port for receiving a lead as shown in FIG. 1 (above) and/or FIG. 3 (below), with individual electrical connectors for each of a plurality of electrodes on the lead(s). Typically, one to four ports are provided, for use with up to four leads, though the present innovation is not limited to a particular lead arrangement.

A communications circuit is also shown at 64. The communications circuit 64 typically includes a resonator, modulator, amplifier and antenna, and may come as a discrete chip. Commercially available chips for Medradio and/or Bluetooth (including Bluetooth Low Energy) can be used, for example. The antenna may be located in the header 62, if desired, to limit signal attenuation due to the housing 50. The communications circuit 64 provides an interface for the device to communicate with external devices including the CP and RC, which may have corresponding circuitry for using the selected communications mode.

The standard approaches to therapy in neuromodulation systems use either current controlled or voltage-controlled therapy generated by a stimulation circuitry 54. The therapy may include, for example, biphasic square waves or monophasic square waves having passive recovery. In general, the amount of current out of an electrode should zero out over time to avoid corrosion at the electrode-tissue interface. For this reason, biphasic pulses, or monophasic pulses with a passive recovery period are typically used. One or several voltage sources may be used, such as with programmable amplifiers or digital to analog conversion circuits that can convert a received therapy command into an analog output voltage, to provide voltage-controlled therapy.

Alternatively, multiple independent current control (MICC) may be used as stimulation circuitry 54. MICC is a stimulus control system that provides a plurality of independently generated output currents that may each have an independent quantity of current. The use of MICC can allow spatially selective fields to be created by therapy outputs. The term “fractionalization” may refer to how the total current issued by the pulse generator via the electrodes is divided up amongst the electrodes of the lead and/or including the pulse generator canister, which can serve as an additional electrode.

Some examples of current or prior versions of IPG circuitry, including in particular the stimulation circuitry 54 but also power 52, I/O 60, and microcontroller 56, as well as planned future examples, may be found in U.S. Pat. No. 10,716,932, the disclosure of which is incorporated herein by reference. Pulse generator circuitry may include that of the various commercially known implantable pulse generators for spinal cord stimulation, Vagus nerve stimulation, and deep brain stimulation as are also well known. Additional examples of circuitry, designs and operation of system devices (IPG, CP, RC, Charger, and ETS, for example) can be found, for example and without limitation, in U.S. Pat. Nos. 6,895,280, 6,181,969, 6,516,227, 6,609,029, 6,609,032, 6,741,892, 7,949,395, 7,244,150, 7,672,734, 7,761,165, 7,974,706, 8,175,710, 8,224,450, and 8,364,278, the disclosures of which are incorporated herein by reference in their entireties.

The circuitry blocks shown in FIG. 2 may be referred to as operational circuitry, and may be described using additional terms specific to particular circuitry functions thereof.

FIG. 3 shows an illustrative spinal cord stimulation SCS system as implanted. In this example, an IPG 70 may be placed near the buttocks or in the abdomen of the patient, with or without a lead extension 72 for coupling to the lead(s) 74 that enter the spinal column. Region 76 at about the level of the lower thoracic or upper lumbar vertebrae may serve as an entry point to the spinal column, where the distal end of the lead 74 with an electrode array may be placed close to the spinal cord 80. Other locations for the IPG 70 and/or lead 74 may be used. For example, sacral nerve stimulation may be performed by positioning the IPG in the lower torso, and extending a lead to near the sacral nerve, as is known in the art, or in the alternative, using a microstimulator. Peripheral nerve stimulation may also be performed, using an IPG and lead positioned at a desired location near the target neural structure, and/or using a microstimulator positioned near the target neural structure. The SCS implementation may include each of the external devices (CP, RC, Charger, ETS) identified in FIG. 1, though not shown in FIG. 3.

Another illustrative a non-limiting example includes an implantable medical device system comprising: an implantable medical device (IMD) (10, 50, 70) comprising operational circuitry including a stimulation output circuitry (54) configured to generate therapy outputs and a controller (56) for controlling the stimulation output circuitry. The controller may be a microcontroller, if desired, but other control structures as described above, including a state machine, may be used. The system also includes a lead (12, 74) extending from the IMD and carrying a plurality of electrodes for delivering stimuli to patient tissue, the lead comprising one or more conductors to electrically couple the electrodes to the IMD. For this example, the controller is configured for using a trigger window (104) and a trigger requirement (112), the trigger window including a trigger window start time, a trigger window end time, and a definition of a first trigger window condition and a first trigger response, and a second trigger window condition and a second trigger response, and the trigger requirement including a trigger requirement start time, a trigger requirement end time, and a definition of a first trigger requirement condition and a first trigger requirement response, and a second trigger requirement condition and a second trigger requirement response; the controller being configured to: deliver a neuromodulation therapy to the patient; determine whether the trigger window start time or the trigger requirement start time has been reached; upon reaching the trigger window start time: initiating observation for the first trigger window condition and the second trigger window condition; in response to observing the first trigger window condition, executing the first trigger response to change the neuromodulation therapy delivered to the patient; in response to observing the second trigger window condition, executing the second trigger response to change the neuromodulation therapy delivered to the patient; and upon reaching the trigger requirement start time: initiating observation for the first trigger requirement condition and the second trigger requirement condition; in response to observing the first trigger requirement condition, executing the first trigger requirement response to change the neuromodulation therapy delivered to the patient; in response to observing the second trigger requirement condition, executing the second trigger requirement response to change the neuromodulation therapy delivered to the patient. FIG. 4 shows an illustrative example.

Additionally or alternatively, the controller is further configured to change a parameter of the neuromodulation therapy upon reaching the trigger window end time in the absence of an observation of the trigger window trigger. Additionally or alternatively, the controller is configured to change the neuromodulation therapy delivered to the patient at the trigger window end time. Additionally or alternatively, the controller is configured to change the neuromodulation therapy delivered to the patient occurs a predetermined length of time after the trigger window end time. Additionally or alternatively, the controller is configured to change the neuromodulation therapy in response to reaching the trigger window end time. Additionally or alternatively, the controller is configured to preserve the neuromodulation therapy in response to reaching the trigger requirement end time. Additionally or alternatively, after reaching the trigger requirement start time, the controller is configured to initiate observation for an indication the trigger requirement trigger is not actively occurring.

Additionally or alternatively, the system further includes a patient device in communication with the IMD, wherein the patient device is configured to communicate a received user input to the IMD, and the controller is configured to observe the trigger window trigger in response to the received user input being communicated from the patient device. Additionally or alternatively, the received user input confirms an expected event occurred. Additionally or alternatively, the received user input confirms an expected event has not occurred.

Additionally or alternatively, the trigger requirement trigger occurs for a predetermined length of time prior to changing the neuromodulation therapy delivered to the patient. Additionally or alternatively, the IMD further includes a motion sensor, and the controller is configured to observe an output of the motion sensor to detect lack of activity of the patient, wherein the trigger requirement trigger is a lack of activity in the patient. Additionally or alternatively, the IMD further includes a motion sensor, and the controller is configured to observe an output of the motion sensor to detect activity of the patient, wherein the trigger requirement trigger is activity in the patient. Additionally or alternatively, the IMD includes a communication circuit (64) configured to communicate wirelessly with an activity monitoring device worn by the patient, and the controller is configured to use data from the activity monitoring device, obtained from the communication circuit, to determine whether a trigger window trigger, or a trigger requirement trigger, has occurred. Additionally or alternatively, the trigger window and the trigger requirement are spaced by a period of time during which changes to the neuromodulation therapy are not dependent on a trigger window trigger or trigger requirement trigger.

Additionally or alternatively, the IMD and lead are configured for use as a spinal cord stimulation system, as illustrated in FIG. 3, with the lead and electrodes adapted for positioning along the spinal cord. Additionally or alternatively, the IMD and lead are configured for use as a deep brain stimulation system, as illustrated in FIG. 1, with the lead and electrodes adapted for positioning inside the cranium, such as by using a directional or segmented lead. In still other alternatives, the IMD and lead may be configured for sacral stimulation, vagus nerve stimulation, peripheral nerve stimulation, functional nerve stimulation, or other treatment.

Each of these non-limiting examples can stand on its own, or can be combined in various permutations or combinations with one or more of the other examples.

The above detailed description includes references to the accompanying drawings, which form a part of the detailed description. The drawings show, by way of illustration, specific embodiments. These embodiments are also referred to herein as “examples.” Such examples can include elements in addition to those shown or described. However, the present inventors also contemplate examples in which only those elements shown or described are provided. Moreover, the present inventors also contemplate examples using any combination or permutation of those elements shown or described (or one or more aspects thereof), either with respect to a particular example (or one or more aspects thereof), or with respect to other examples (or one or more aspects thereof) shown or described herein.

In the event of inconsistent usages between this document and any documents so incorporated by reference, the usage in this document controls.

In this document, the terms “a” or “an” are used, as is common in patent documents, to include one or more than one, independent of any other instances or usages of “at least one” or “one or more.” Moreover, in the claims, the terms “first,” “second,” and “third,” etc. are used merely as labels, and are not intended to impose numerical requirements on their objects.

Method examples described herein can be machine or computer-implemented at least in part. Some examples can include a computer-readable medium or machine-readable medium encoded with instructions operable to configure an electronic device to perform methods as described in the above examples. An implementation of such methods can include code, such as microcode, assembly language code, a higher-level language code, or the like. Such code can include computer readable instructions for performing various methods. The code may form portions of computer program products. Further, in an example, the code can be tangibly stored on one or more volatile, non-transitory, or non-volatile tangible computer-readable media, such as during execution or at other times. Examples of these tangible computer-readable media can include, but are not limited to, hard disks, removable magnetic or optical disks, magnetic cassettes, memory cards or sticks, random access memories (RAMs), read only memories (ROMs), and the like.

The above description is intended to be illustrative, and not restrictive. For example, the above-described examples (or one or more aspects thereof) may be used in combination with each other. Other embodiments can be used, such as by one of ordinary skill in the art upon reviewing the above description.

The Abstract is provided to comply with 37 C.F.R. § 1.72(b), to allow the reader to quickly ascertain the nature of the technical disclosure. It is submitted with the understanding that it will not be used to interpret or limit the scope or meaning of the claims.

Also, in the above Detailed Description, various features may be grouped together to streamline the disclosure. This should not be interpreted as intending that an unclaimed disclosed feature is essential to any claim. Rather, innovative subject matter may lie in less than all features of a particular disclosed embodiment. Thus, the following claims are hereby incorporated into the Detailed Description as examples or embodiments, with each claim standing on its own as a separate embodiment, and it is contemplated that such embodiments can be combined with each other in various combinations or permutations. The scope of the protection should be determined with reference to the appended claims, along with the full scope of equivalents to which such claims are entitled.

What is claimed is:

1. An implantable medical device system comprising:
an implantable medical device (IMD) comprising operational circuitry including a stimulation output circuitry configured to generate therapy outputs and a controller for controlling the stimulation output circuitry; and
a lead extending from the IMD and carrying a plurality of electrodes for delivering stimuli to patient tissue, the lead comprising one or more conductors to electrically couple the electrodes to the IMD;
wherein the controller is configured for using a trigger window and a trigger requirement, the trigger window including a trigger window start time, a trigger window end time, and a definition of a trigger window trigger, and the trigger requirement including a trigger requirement start time, a trigger requirement end time, and a definition of a trigger requirement trigger; and the controller is configured to:
deliver a neuromodulation therapy to the patient using the plurality of electrodes;
determine whether the trigger window start time or the trigger requirement start time has been reached;
upon reaching the trigger window start time:
initiate observation for the trigger window trigger;
if the trigger window trigger is observed, change the neuromodulation therapy delivered to the patient; and, if not:
upon reaching the trigger requirement start time:
initiate observation for the trigger requirement trigger; and
if the trigger requirement trigger is observed, changing the neuromodulation therapy delivered to the patient.

2. The system of claim 1, wherein the controller is further configured to change a parameter of the neuromodulation therapy upon reaching the trigger window end time in the absence of an observation of the trigger window trigger.

3. The system of claim 2, wherein the controller is configured to change the neuromodulation therapy delivered to the patient at the trigger window end time.

4. The system of claim 2, wherein the controller is configured to change the neuromodulation therapy delivered to the patient occurs a predetermined length of time after the trigger window end time.

5. The system of claim 2, wherein the controller is configured to change the neuromodulation therapy in response to reaching the trigger window end time.

6. The system of claim 1, wherein the controller is configured to preserve the neuromodulation therapy in response to reaching the trigger requirement end time.

7. The system of claim 1, wherein after reaching the trigger requirement start time, the controller is configured to initiate observation for an indication the trigger requirement trigger is not actively occurring.

8. The system of claim 1, further comprising a patient device in communication with the IMD, wherein the patient device is configured to communicate a received user input to the IMD, and the controller is configured to observe the trigger window trigger in response to the received user input being communicated from the patient device.

9. The system of claim 1, wherein the controller is configures so that the trigger requirement trigger occurs for a predetermined length of time prior to changing the neuromodulation therapy delivered to the patient.

10. The system of claim 1, wherein the IMD further includes a motion sensor, and the controller is configured to observe an output of the motion sensor to detect lack of activity of the patient, wherein the trigger requirement trigger is an activity or a a lack of activity in the patient.

11. The system of claim 1, wherein the definition of a trigger window trigger includes a definition of a first trigger window condition and a first trigger response, and a definition of a second trigger window condition and a second trigger response; and the controller is further configured to:
initiate observation for the trigger window trigger by observing for each of the first trigger window condition and the second trigger window condition; and
change a neuromodulation therapy delivered to the patient in response to observation of the trigger window trigger by either:
in response to observing the first trigger window condition, executing the first trigger response to change the neuromodulation therapy; or
in response to observing the second trigger window condition, executing the second window trigger response to change the neuromodulation therapy.

12. The system of claim 1, wherein the definition of a trigger requirement trigger includes a definition of a first trigger requirement condition and a first trigger requirement response, and a definition of a second trigger requirement condition and a second trigger requirement response; and the controller is further configured to
initiate observation for the trigger requirement trigger by observing for each of the first trigger requirement condition and the second trigger requirement condition; and
change the neuromodulation delivered to the patient therapy in response to observation of the trigger requirement trigger by either:
in response to observing the first trigger requirement condition, executing the first trigger requirement response to change the neuromodulation therapy; or
in response to observing the second trigger requirement condition, executing the second trigger requirement response to change the neuromodulation therapy.

13. A method of delivering a neuromodulation therapy comprising:
setting, for a neuromodulation system implanted in a patient, at least one trigger window, the at least one trigger window including a trigger window start time, a trigger window end time, and a definition of a trigger window trigger, and
upon reaching the trigger window start time:
initiating observation for the trigger window trigger;
in response to observation of the trigger window trigger, changing a neuromodulation therapy delivered to the patient.

14. The method of claim 13, further comprising setting, for the neuromodulation system implanted in the patient, at least one trigger requirement, the trigger requirement including a trigger requirement start time, a trigger requirement end time, and a definition of a trigger requirement trigger.

15. The method of claim 14, further comprising
upon reaching the trigger requirement start time:
initiating observation for the trigger requirement trigger;
in response to observation of the trigger requirement trigger, changing the neuromodulation therapy delivered to the patient; and
in response to reaching the trigger requirement end time, preserving the neuromodulation therapy delivered to the patient.

16. The method of claim 14, in response to reaching the trigger window end time, changing the neuromodulation therapy delivered to the patient.

17. The method of claim 14, in response to reaching the trigger requirement end time, preserving the neuromodulation therapy delivered to the patient.

18. The method of claim 14, wherein after reaching the trigger requirement start time:
initiating observation for an indication the trigger requirement trigger is not actively occurring;
wherein observation of the indication the trigger requirement trigger is not actively occurring initiates a hold off period.

19. The method of claim 13, wherein the definition of a trigger window trigger includes a definition of a first trigger window condition and a first trigger response, and a definition of a second trigger window condition and a second trigger response;
the step of initiating observation for the trigger window trigger includes observing for each of the first trigger window condition and the second trigger window condition;
the step of changing a neuromodulation therapy delivered to the patient in response to observation of the trigger window trigger comprises either:
in response to observing the first trigger window condition, executing the first trigger response to change the neuromodulation therapy; or
in response to observing the second trigger window condition, executing the second window trigger response to change the neuromodulation therapy.

20. A method of delivering a neuromodulation therapy comprising:
setting, for a neuromodulation system implanted in a patient, a trigger window and a trigger requirement:
the trigger window including a trigger window start time, a trigger window end time, and a definition of a trigger window trigger, and
the trigger requirement including a trigger requirement start time, a trigger requirement end time, and a definition of a trigger requirement trigger;
upon reaching the trigger window start time:
initiating observation for the trigger window trigger;
in response to observation of the trigger window trigger, changing a neuromodulation therapy delivered to the patient;
in response to reaching the trigger window end time, changing the neuromodulation therapy delivered to the patient; and
upon reaching the trigger requirement start time:
initiating observation for the trigger requirement trigger;
in response to observation of the trigger requirement trigger, changing the neuromodulation therapy delivered to the patient; and
in response to reaching the trigger requirement end time, preserving the neuromodulation therapy delivered to the patient.
